(19) United States
(12) Patent Application Publication
(10) Pub. No.: US 2022/0249402 A1
(43) Pub. Date: Aug. 11, 2022

(54) DIETARY SUPPLEMENT AND MEDICAMENT

(71) Applicant: NUTRIOMICS LIMITED, Te Awamutu (NZ)

(72) Inventor: Aydin Berenjian, State College, PA (US)

(21) Appl. No.: 17/446,117

(22) Filed: Aug. 26, 2021

(30) Foreign Application Priority Data
Feb. 10, 2021 (NZ) 772850
Feb. 10, 2021 (NZ) 772851

(51) Int. Cl. A61K 31/122; A61K 35/618; A61K 35/57; A61K 31/593; A61K 31/375; A61K 36/9068; A61K 33/06; A61P 19/02

(52) U.S. Cl. CPC A61K 31/122; A61K 35/618; A61K 35/57; A61P 19/02; A61K 31/375; A61K 36/9068; A61K 33/06; A61K 31/593

(57) ABSTRACT

The present invention is directed to a dietary supplement comprising about 120 mcg of a trans form of Menaquinone-7 (MK-7). In some embodiments, the dietary supplement may further comprise a combination of green-lipped mussel powder and eggshell membrane powder. In other embodiments, the dietary supplement may comprise vitamin D3, vitamin C, and Ginger roots extract.

### CROSS-REFERENCES TO RELATED APPLICATIONS

This application claims benefit of New Zealand patent number 772,850, filed Feb. 10, 2021, and of New Zealand patent number 772,851, filed Feb. 10, 2021, the specifications of which are incorporated herein in their entirety by reference.

### TECHNICAL FIELD

The present invention is in the field of dietary supplements with a specific dosage of key nutrients to preserve and help with growth, and repair of joint connective tissue.

### BACKGROUND OF THE INVENTION

Treatment of joint pain depends on its cause and severity. Non-steroidal anti-inflammatory drugs (NSAIDs), steroids or even surgical intervention can solve the pain and limit further damage to the joints, but they all pose potential side effects and are almost ineffective for the promotion of joint tissue repair. Nutritional supplements can potentially help the joint health by stimulating the growth, repair and maintenance of bone and joint connective tissue.

One class of supplements includes components of joint connective tissue such as collagen, glucosamine, hyaluronic acid, and chondroitin. Other supplements act as catalysts or supply raw materials for bone and connective tissue synthesis these are mainly S-adenosylmethionine (SAM), methylsulfonylmethane (MSM), and other vitamins and minerals such as Vitamin C, manganese, magnesium, zinc, calcium, iron, and Vitamin B12.

Compositions containing glucosamine are known to be beneficial to both humans and animals that suffer from osteoarthritis pain. Since glucosamine is a precursor for glycosaminoglycans, and glycosaminoglycans are a major component of joint cartilage, supplemental glucosamine may help to rebuild cartilage and treat arthritis. Commonly sold forms of glucosamine are glucosamine sulfate and glucosamine hydrochloride. Glucosamine is often sold in combination with other supplements such as chondroitin sulfate and methylsulfonylmethane. Generally, vitamin C is needed together with glucosamine sulfate.

Chondroitin sulfate is a sulfated glycosaminoglycan (GAG) composed of a chain of alternating sugars (N-acetyl-galactosamine and glucuronic acid). It is usually found attached to proteins as part of a proteoglycan. Chondroitin sulfate is a major structural component of cartilage and provides much of its resistance to compression. Most of these supplements derive the Chondroitin Sulfate from bovine cartilage or velvet deer antler or shark cartilage. Avocado/soybean unsaponifiables (ASU) are natural vegetable extracts made from avocado and soybean oils, consisting of the leftover fraction (approximately 1%) that cannot be made into soap after saponification. ASU is composed of one third avocado and two thirds soybean unsaponifiables. The major components of ASU are phytosterols β-sitosterol, campesterol, and stigmasterol, which are rapidly incorporated into cells. The sterol contents of ASU preparations are the primary contributors to biological activity in articular chondrocytes. Preclinical in vitro and in vivo studies have demonstrated that ASUs have beneficial effects on promoting cartilage repair.

Collagen is an essential and major component of muscles, tendons, cartilage, ligaments, joints and blood vessels in humans or animals. Methylsulfonylmethane (MSM, or dimethylsulfone) is an organic sulfur compound belonging to a class of chemicals known as sulfones. It occurs naturally in some primitive plants and is present in small amounts in many foods and beverages. MSM is also known as dimethylsulfone, or DMSO2, a name that reflects its close chemical relationship to dimethyl sulfoxide (DMSO), which differs only in the oxidation state of the sulphur atom. MSM is the primary metabolite of DMSO in humans, and it shares some of the properties of DMSO.

It can be seen that tissue maintenance and building is a complex process that requires range of nutrients at the same time to provide synergistic effect to preserve and rebuild joint tissue. A need, therefore, exists for a new type of nutritional supplement that can be utilized to improve overall joint health.

### DETAILED DESCRIPTION OF THE INVENTION

The term “joint disorders” can be taken to include but not be limited to

a) osteoarthritis,

b) joint effusion,

c) joint erosion,

d) joint inflammation and pain,

e) joint calcification

f) the reduction or inhibition of metabolic activity of chondrocytes,

g) the reduction or inhibition of enzymes that degrade cartilage,

Menaquinone-7 (MK-7), sub-type of Vitamin K2 (Menaquinones), can exist as cis and trans isomers. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”),

The chemical structure of MK-7 influences its ability to interact with subcellular structures, and thus determines its biological activity. Therefore, only the all-trans form of MK-7 is biologically significant. Among the various aspects of the invention, the dietary supplement includes the trans form of MK-7 that can exert an active role in joint cells metabolism.

Another aspect of the invention encompasses a dietary supplement having a balanced mixture of key compounds including trans form of MK-7, green-lipped mussel powder, eggshell membrane powder, vitamin D3, vitamin C, and ginger root extract that together promote the growth, repair, and maintenance of mammalian joint connective tissue. The supplement may be administered as a paste, chewable flavored tablet, capsule, or a powder appointed to be admixed with the food products. In preferred embodiments, the supplement properly will include additional excipients that will aid to balance metabolic needs for efficacy.

In accordance with one aspect of the present invention, the dietary supplements may be administered to a mammalian subject to prevent several joint disorders or indications, including but not be limited to

h) osteoarthritis,

i) joint effusion,

j) joint erosion,

k) joint inflammation and pain,

I) the reduction or inhibition of metabolic activity of chondrocytes,

m) the reduction or inhibition of enzymes that degrade cartilage,

n) the reduction or inhibition of the production of hyaluronic acid.

The dietary supplements may also be administered to a mammalian subject to decrease degradation of articular cartilage or disorders or indications resulting from degradation of articular cartilage. The dietary supplement includes a mixture of trans form of MK-7, green-lipped mussel powder, eggshell membrane powder, vitamin D3, vitamin C, and ginger root extract. The dietary supplement can, in accordance with generally known methods, be formulated to meet the needs of several mammalian subjects. It is contemplated, that one or more excipients being used during the formulation without departing from the scope of the invention.

A variety of commonly used excipients in dietary supplement formulations may be selected on the basis of compatibility with the active ingredients. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste masking agents, an effervescent disintegration agent, and combinations of any of these agents.

In one embodiment, the excipient is a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof. The polypeptide may be any arrangement of amino acids.

In another embodiment, the excipient may be a filler. Suitable fillers include carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate; magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch; pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In another embodiment; the excipient may be an effervescent disintegrant. By way of non-limiting example, suitable effervescent disintegrants include sodium bicarbonate in combination with citric acid and sodium bicarbonate in combination with tartaric acid. The excipient may comprise a preservative. Suitable examples of preservatives include antioxidants, such as a-tocopherol or ascorbate, and antimicrobials, such as parabens, chlorobutanol or phenol.

In another embodiment, the excipient may include a diluent. Diluents suitable for use include pharmaceutically acceptable saccharide such as sucrose, dextrose, lactose, microcrystalline cellulose, fructose, xylitol, and sorbitol; polyhydric alcohols; a starch; pre-manufactured direct compression diluents; and mixtures of any of the foregoing. The excipient may be a dispersion enhancer. Suitable dispersants may include starch, alginic acid, polyvinylpyrrolidones, guar gum, kaolin; bentonite, purified wood cellulose, sodium starch glycolate, isomorphous silicate, and microcrystalline cellulose as high HLB emulsifier surfactants.

The dietary supplements detailed herein includes trans form of vitamin MK7 (120 mcg), green-lipped mussel powder (Perna canaliculus) (552.7 mg), eggshell membrane powder (500 mg), vitamin D3 (800 IU), vitamin C (calcium ascorbate dihydrate) (121.72 mg) and Ginger roots extract (1.6 mg). Suitable dosage forms include a tablet, including a suspension tablet, a chewable tablet, an effervescent tablet or caplet; a pill; a powder such as a sterile packaged powder, a dispensable powder, and an effervescent powder; a capsule including both soft or hard gelatin capsules or non-animal derived polymers, such as hydroxypropyl methylcellulose capsules or pullulan; a lozenge; a sachet; a sprinkle; a reconstitutable powder or shake; a troche; pellets; granules; liquids; suspensions; emulsions; or semisolids and gels.

Alternatively, the dietary supplement may be incorporated into a food product or powder for mixing with a liquid, or administered orally after only mixing with a non-foodstuff liquid.

Example 1—A supplement regime in accordance with the present invention includes the following.

| Component | Dosage | |
|---|---|---|
| Trans form of MK-7 | 120 | mcg/day |
| Green-lipped mussel powder | 552.7 | mg/day |
| Eggshell membrane powder | 500 | mg/day |
| Vitamin D3 | 800 | IU/day |
| Calcium ascorbate dihydrate | 121.72 | mg/day |
| Ginger root extract | 1.6 | mg/day |

Example 2—NADPH oxidase activity involves in reducing molecular oxygen resulting in ROS production and superoxide anion. ROS formation was measured using human chondrocyte cells. Culture media composed of trans form of vitamin MK7 (120 mcg), green-lipped mussel powder (Perna canaliculus) (552.7 mg), eggshell membrane powder (500 mg), vitamin D3 (800 IU), vitamin C (calcium ascorbate dihydrate) (121.72 mg) and Ginger roots extract (1.6 mg). After 96 hours of incubation, tetracycline was used to measure the NADPH oxidase activity by chemiluminescence. Impact of Nutraceutical complex on NADPH oxidase activity is shown in FIG. 1. It is clear that the NADPH oxidase activity significantly decreased after 96 hours pre-incubation. In the presence of the nutraceutical complex 30% reduction in ROS level was observed as compared to the control samples. This behaviour demonstrates the synergistic effect of the ingredients complex in decreasing the NADPH oxidase activity and ROS production.

Example 3—Chondrocyte cells were incubated for seven days in Dulbecco's Modified Eagle with and without 2 ng/ml of IL-1β after 96 hours pre-incubation with nutraceutical complex composed of trans form of vitamin MK7 (120 mcg), green-lipped mussel powder (Perna canaliculus) (552.7 mg), eggshell membrane powder (500 mg), vitamin D3 (800 IU), vitamin C (calcium ascorbate dihydrate) (121.72 mg) and Ginger roots extract (1.6 mg). Cells were separated and placed in Dulbecco's Modified Eagle containing 10% foetal bovine serum. Finally, alive cells were counted using trypan blue assay method. IL-1β upregulation leads to disturbing of the cell cycle, cell senescence, and finally cell apoptosis. Impact of Nutraceutical complex on chondrocyte viability is shown in FIG. 2. Based on the results, in the presence of IL-1β, a meaningful decrease in chondrocyte cells survival was observed. However, chondrocyte survival was improved by 4.4-fold in the culture media composed of trans form of vitamin MK7 (120 mcg), green-lipped mussel powder (Perna canaliculus) (552.7 mg), eggshell membrane powder (500 mg), vitamin D3 (800 IU), vitamin C (calcium ascorbate dihydrate) (121.72 mg) and Ginger roots extract (1.6 mg) as compared to control samples.

Example 4—MGP expression in the presence of culture media composed of trans form of vitamin MK7 (120 mcg), green-lipped mussel powder (Perna canaliculus) (552.7 mg), eggshell membrane powder (500 mg), vitamin D3 (800 IU), vitamin C (calcium ascorbate dihydrate) (121.72 mg) and Ginger roots extract (1.6 mg) were measured as compared to control samples. Results were reported as fold change in gene expression relative to control conditions as shown in FIG. 3. MGP expression was increased by 33% after treatment with the nutraceutical complex in comparison to the control samples. This observation demonstrates the synergistic effect of the nutraceutical complex in stimulating the level of mRNA encoding MGP which could be related to a transcriptional activation of the MGP gene. MGP is a protein that is highly expressed by chondrocytes. MGP gene expression can be regulated by several mechanisms that would have the potential to become genomic biomarkers for the prediction of soft tissue calcification as well as its progression.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a graph illustrating the impact of the nutraceutical complex on NADPH oxidase activity.

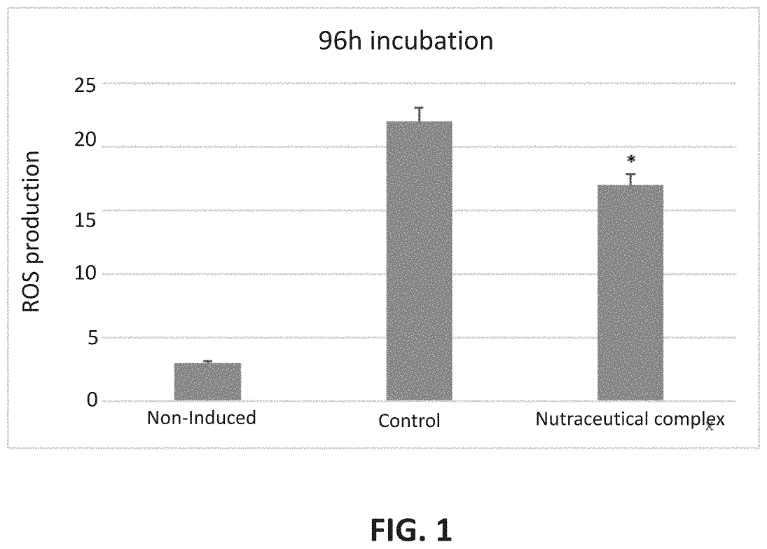

FIG. 2 shows a graph illustrating the impact of the nutraceutical complex on chondrocyte viability.

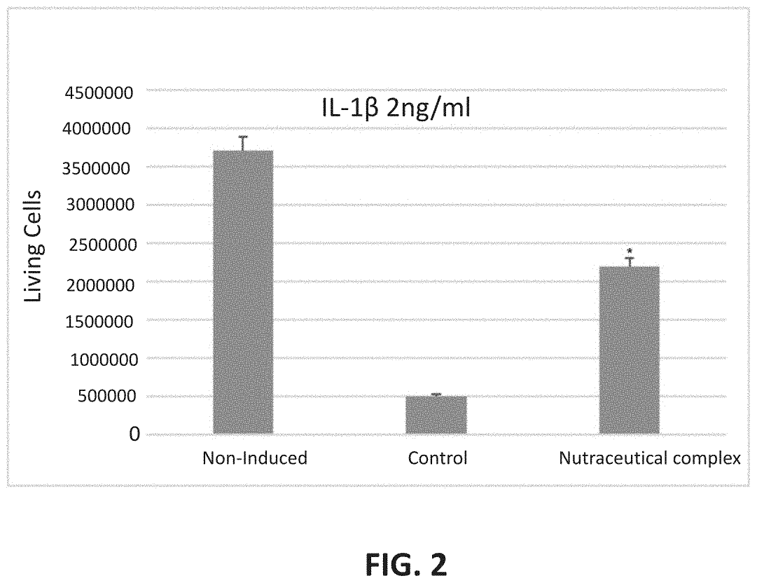

FIG. 3 shows a graph illustrating the fold change in MGP expression relative to control conditions.

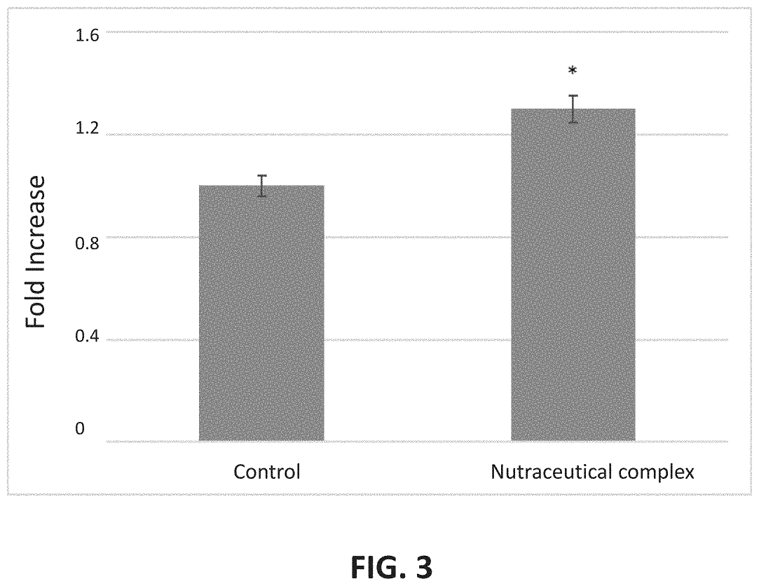

### REFERENCES

1. Vos, T., et al., Global, regional, and national incidence, prevalence, and years lived with disability for 328 diseases and injuries for 195 countries, 1990-2016: A systematic analysis for the Global Burden of Disease Study 2016. The Lancet, 2017. 390(10100): p. 1211-1259.
2. Jindai, K., et al., Multimorbidity and functional limitations among adults 65 or older, NHANES 2005-2012. Preventing Chronic Disease, 2016. 13(11): p. 1-11.
3. Lotz, M. and R. F. Loeser, Effects of aging on articular cartilage homeostasis. Bone, 2012. 51(2): p. 241-248.
4. Mitsuyama, H., et al., Calcification of human articular knee cartilage is primarily an effect of aging rather than osteoarthritis. Osteoarthritis and Cartilage, 2007. 15(5): p. 559-565.
5. Pritzker, K. P. H., Counterpoint: Hydroxyapatite crystal deposition is not intimately involved in the pathogenesis and progression of human osteoarthritis, Current Rheumatology Reports, 2009. 11(2): p. 148-153.
6. Mobasheri, A., et al., Chondrosenescence: Definition, hallmarks and potential role in the pathogenesis of osteoarthritis. Maturitas, 2015. 80(3): p. 237-244.
7. Greene, M. A. and R. F. Loeser, Aging-related inflammation in osteoarthritis. Osteoarthritis and Cartilage, 2015. 23(11): p. 1966-1971.
8. Van Der Kraan, P., C. Matta, and A. Mobasheri, Age-Related Alterations in Signaling Pathways in Articular Chondrocytes: Implications for the Pathogenesis and Progression of Osteoarthritis—A Mini-Review. Gerontology, 2016. 63(1): p. 29-35.
9. Lepetsos, P. and A. G. Papavassiliou, ROS/oxidative stress signaling in osteoarthritis. Biochimica et Biophysica Acta—Molecular Basis of Disease, 2016. 1862(4): p. 576-591.
10. Bedard, K. and K. H. Krause, The NOX family of ROS-generating NADPH oxidases: Physiology and pathophysiology. Physiological Reviews, 2007. 87(1): p. 245-313.
11. Rousset, F., et al., Herne Oxygenase-1 Regulates Matrix Metalloproteinase MMP-1 Secretion and Chondrocyte Cell Death via Nox4 NADPH Oxidase Activity in Chondrocytes. PLoS ONE, 2013. 8(6),
12. Goldring, M. B., et al., Defining the roles of inflammatory and anabolic cytokines in cartilage metabolism. Annals of the Rheumatic Diseases, 2008. 67(SUPPL. 3): p. iii75-iii82.
13. Magne, D., et al., Phosphate is a specific signal for ATDC5 chondrocyte maturation and apoptosis-associated mineralization: Possible implication of apoptosis in the regulation of endochondral ossification. Journal of Bone and Mineral Research, 2003. 18(8): p. 1430-1442.
14. Terkeltaub, R. A., What does cartilage calcification tell us about osteoarthritis? Journal of Rheumatology, 2002. 29(3): p. 411-415.
15. Bannuru, R. R., et al., OARSI guidelines for the non-surgical management of knee, hip, and polyarticular osteoarthritis. Osteoarthritis and Cartilage, 2019. 27(11): p. 1578-1589.
16. Shea, M. K. and S. L. Booth, Vitamin K, Osteoarthritis, and Joint Pain, in Nutritional Modulators of Pain in the Aging Population. 2017. p. 225-233.
17. Berkner, K. L., Vitamin K-Dependent Carboxylation, in Vitamins and Hormones. 2008. p. 131-156.
18. Lindholt, J. S., et al., Effects of menaquinone-7 supplementation in patients with aortic valve calcification: Study protocol for a randomised controlled trial. BMJ Open, 2018. 8(8).
19. Wei, F. F., et al., Vitamin K-Dependent Matrix Gla Protein as Multifaceted Protector of Vascular and Tissue Integrity. Hypertension (Dallas, Tex.: 1979), 2019. 73(6): p. 1160-1169.
20. Lal, N. and A. Berenjian, Cis and trans isomers of the vitamin menaquinone-7: which one is biologically significant? Applied Microbiology and Biotechnology, 2020, 104(7): p. 2765-2776.
21. Brien, S., et al., Systematic review of the nutritional supplement Perna Canaliculus (green-lipped mussel) in the treatment of osteoarthritis. QJM, 2008. 101(3): p. 167-179.
22. Rialland, P., et al., Effect of a diet enriched with green-lipped mussel on pain behavior and functioning in dogs with clinical osteoarthritis. Canadian Journal of Veterinary Research, 2013. 77(1): p. 66-74.
23. Vojinovic, J., et al., European multicentre pilot survey to assess vitamin D status in rheumatoid arthritis patients and early development of a new Patient Reported Outcome questionnaire (D-PRO). Autoimmunity Reviews, 2017. 16(5): p. 548-554.
24. Zhang, F. F., et al., Vitamin D deficiency is associated with progression of knee osteoarthritis. The Journal of nutrition, 2014. 144(12): p. 2002-2008.
25. Lee, Y. H. and S. C. Bae, Vitamin D level in rheumatoid arthritis and its correlation with the disease activity: A meta-analysis. Clinical and Experimental Rheumatology, 2016. 34(5): p. 827-833.
26. Mabey, T. and S. Honsawek, Role of Vitamin D in Osteoarthritis: Molecular, Cellular, and Clinical Perspectives. International Journal of Endocrinology, 2015. 2015.
27. Grover, A. K. and S. E. Samson, Benefits of antioxidant supplements for knee osteoarthritis: Rationale and reality, Nutrition Journal, 2016. 15(1).
28. DePhillipo, N. N., et al., Efficacy of Vitamin C Supplementation on Collagen Synthesis and Oxidative Stress After Musculoskeletal Injuries: A Systematic Review. Orthopaedic journal of sports medicine, 2018. 6(10): p. 2325967118804544-2325967118804544.
29. Bolognesi, G., et al., Movardol® (N-acetylglucosamine, Boswellia serrata, ginger) supplementation in the management of knee osteoarthritis: preliminary results from a 6-month registry study. European review for medical and pharmacological sciences, 2016. 20(24): p. 5198-5204.
30. Zhang, L., et al., New insight into the Nox4 subcellular localization in HEK293 cells: First monoclonal antibodies against Nox4. Biochimie, 2011. 93(3): p. 457-468.
31. Grange, L., et al., NAD(P)H oxidase activity of Nox4 in chondrocytes is both inducible and involved in collagenase expression. Antioxidants and Redox Signaling, 2006. 8(9-10): p. 1485-1496.
32. Serrander, L., et al., NOX4 activity is determined by mRNA levels and reveals a unique pattern of ROS generation. Biochemical Journal, 2007. 406(1): p. 105-114.
33. Dozin, B., et al., Response of young, aged and osteoarthritic human articular chondrocytes to inflammatory cytokines: Molecular and cellular aspects. Matrix Biology, 2002. 21(5): p. 449-459.
34. Shanahan, C. M., et al., High expression of genes for calcification-regulating proteins in human atherosclerotic plaques. Journal of Clinical Investigation, 1994. 93(6): p. 2393-2402.
35. Shanahan, C. M., et al., The role of Gla proteins in vascular calcification. Critical Reviews in Eukaryotic Gene Expression, 1998. 8(3-4): p. 357-375.
36. Luo, G., et al., Spontaneous calcification of arteries and cartilage in mice lacking matrix GLA protein. Nature, 1997. 386(6620): p. 78-81.
37. Price, P. A., et al., Excessive mineralization with growth plate closure in rats on chronic warfarin treatment. Proceedings of the National Academy of Sciences of the United States of America, 1982. 79(24 I): p. 7734-7738.
38. Bjørklund, G., et al., The role of matrix gla protein (MGP) in vascular calcification. Current Medicinal Chemistry, 2020. 27(10): p. 1647-1660.

1. A dietary supplement comprising about 120 mcg of a trans form of Menaquinone-7 (MK-7).

2. The dietary supplement of claim 1, further comprising a combination of green-lipped mussel powder and eggshell membrane powder.

3. The dietary supplement of claim 2, wherein the dietary supplement comprises about 552.7 mg of green-lipped mussel powder and about 500 mg of eggshell membrane powder

4. The dietary supplement of claim 1, further comprising vitamin D3, vitamin C, and Ginger roots extract.

5. The dietary supplement of claim 4, wherein the dietary supplement comprises about 800 IU of vitamin D3, about 121.72 mg of vitamin C, and about 1.6 mg of Ginger roots extract.

6. A medicament for the therapeutic or prophylactic treatment of joint conditions comprising a combination of the trans form of Menaquinone-7 (MK7), green-lipped mussel powder, eggshell membrane powder, vitamin D3, vitamin C and Ginger roots extract.

7. A method for promoting at least one of pain relief, growth, repair, or maintenance of bone or joint tissue in a mammalian subject, the method comprising administering to the mammalian subject the medicament of claim 6.

8. The method of claim 7, wherein the medicament further comprises at least one ingredient selected from the group consisting of excipients.

9. The method of claim 7, wherein the mammalian subject is selected from the group consisting of animals and humans.

10. The method of claim 7, wherein the mammalian subject has a joint-related indication.

11. The method of claim 10, wherein the joint-related indication is selected from the group consisting of osteoarthritis, rheumatoid arthritis, psoriatic arthritis, joint effusion, joint inflammation or pain, synovitis, lameness, post-operative arthroscopic surgery, deterioration of proper joint function, the inhibition of metabolic activity of chondrocytes, and the inhibition of the production of hyaluronic acid.
